(19) United States
(12) Patent Application Publication
(10) Pub. No.: US 2006/0265054 A1
(43) Pub. Date: Nov. 23, 2006

(54) Filament Based Prosthesis

(76) Inventors: Skott E. Greenhalgh, Glenside, PA (US); Robert S. Schwartz, Rochester, MN (US); Robert A. Van Tassel, Excelsior, MN (US); Tom Molz, Warrington, PA (US)

Correspondence Address: INSKEEP INTELLECTUAL PROPERTY GROUP, INC, 2281 W. 190TH STREET, SUITE 200, TORRANCE, CA, 90504, US

(21) Appl. No.: 11/460,911

(22) Filed: Jul. 28, 2006

Related U.S. Application Data
(63) Continuation of application No. 10/856,893, filed on May 27, 2004.
(60) Provisional application No. 60/474,682, filed on May 29, 2003.
(60) Provisional application No. 60/489,126, filed on Jul. 21, 2003.

(51) Int. Cl. A61F 2/06

(52) U.S. Cl. 623/1.31

(57) ABSTRACT

The present invention includes a prosthesis device composed of a plurality of filaments engaged together to self expand against the inner surface of a vessel. In this respect a pocket is created between the prosthesis and the vessel walls which prevent plaque and other debris from escaping downstream to potentially cause complications.

### CROSS REFERENCE TO RELATED APPLICATIONS

This application claims the benefit of U.S. Provisional Application 60/474,682, entitled Mesh Based Integral Embolic Stent And PTCA Protection, filed May 29, 2003, and U.S. Provisional Application 60/489,126, entitled Mesh Based Integral Embolic Stent And PTCA Protection—Version II, filed Jul. 21, 2003, which are both hereby incorporated by reference.

### BACKGROUND OF THE INVENTION

Currently, minimally invasive surgical techniques are practiced to treat various disease conditions of the cardiovascular system of the human body such as a stenosis, arteriosclerosis or atherosclerosis. For example, popular minimally invasive treatments include balloon angioplasty, thrombolysis, and stent placement.

Although minimally invasive techniques are often safer than more invasive disease treatments, they risk dislodging plaque, also referred to as emboli, built up along the inner walls of a patient's blood vessel. Once dislodged, the plaque may result in possibly serious complications downstream of the treatment site. For example, treatment of a stenosis in a carotid artery can result in ischemic complications and possibly embolic stroke.

To reduce the risk of treatment related complications, many prior art blood filters have been developed. Most of the catheter-based blood filters in the prior art involve deploying an expandable filter downstream of the treatment portion of the catheter (e.g. angioplasty balloon or stent). Therefore, if plaque or other debris is dislodged during a treatment procedure, the blood filter stops the plaque from moving to other regions of the body. Such designs can be seen in example U.S. Pat. Nos. 5,827,324, 6,027,520, or 6,142,987, the contents of each of which are hereby incorporated by reference.

Although the prior art downstream filter designs may block most dislodged plaque, some fail to completely expand through the entire diameter of the blood vessel, providing an opportunity for smaller pieces of plaque to slip by. Further, these prior art filter designs often retract back into the catheter, during which time captured plaque may escape past the filter.

Another solution to emboli related complications can be seen in U.S. Pat. No. 6,312,463, the contents of which are hereby incorporated by reference. The prior art design of this patent describes a fabric having anchoring elements which urge the fabric to expand against the vessel walls of a treatment site prior to deployment of a stent. However, since the fabric requires an anchoring element to expand, it takes up valuable space within the diameter of the vessel. Further, such a combination does not easily conform to structural irregularities within the vessel.

### OBJECTS AND SUMMARY OF THE INVENTION

It is an object of the present invention to overcome the above stated limitations of the prior art.

It is a further object of the present invention to provide a self expanding prosthesis.

It is a further object of the present invention to provide a prosthesis that better protects a patient from emboli related complications.

The above stated objects are achieved with the present invention, which includes a prosthesis device composed of a plurality of filaments engaged together to self expand against the inner surface of a vessel. In this respect a pocket is created between the prosthesis and the vessel walls which prevent plaque and other debris from escaping downstream to potentially cause complications.

### BRIEF DESCRIPTION OF THE DRAWINGS

FIG. 1 illustrates a side view of a prosthesis device according to the present invention;

FIG. 2 illustrates a side view of the prosthesis device of FIG. 1;

FIGS. 3A and 3B illustrate side views of the prosthesis device of FIG. 1;

FIG. 4A illustrates a side view of a vessel;

FIGS. 4B and 4C illustrate side views of a prosthesis device according to the present invention;

FIGS. 5A-5C illustrate side views of a prosthesis device according to the present invention;

FIGS. 6A-6B illustrate side views of a prosthesis device according to the present invention;

FIG. 7A illustrates a side view of a vessel;

FIGS. 7B and 7C illustrate side views of a prosthesis device according to the present invention;

FIGS. 8A-8C illustrate side views of a prosthesis device according to the present invention;

FIG. 9 illustrates a side view of a prosthesis device according to the present invention;

FIGS. 10A-10C illustrates side views of a prosthesis device according to the present invention;

FIG. 11 illustrates a side view of a prosthesis device according to the present invention;

FIGS. 12 and 13 illustrate a side view of a prosthesis device according to the present invention;

FIG. 14 illustrates a perspective view of a prosthesis device with micro pleats according to the present invention;

FIG. 15 illustrates a perspective view of a prosthesis device with micro pleats according to the present invention;

FIG. 16 illustrates a side view of a prosthesis device according to the present invention;

FIG. 17 illustrates a side view of a prosthesis device according to the present invention; and

FIG. 18 illustrates a micrograph of the prosthesis of FIG. 17.

### DETAILED DESCRIPTION OF THE INVENTION

Self Expanding Prosthesis

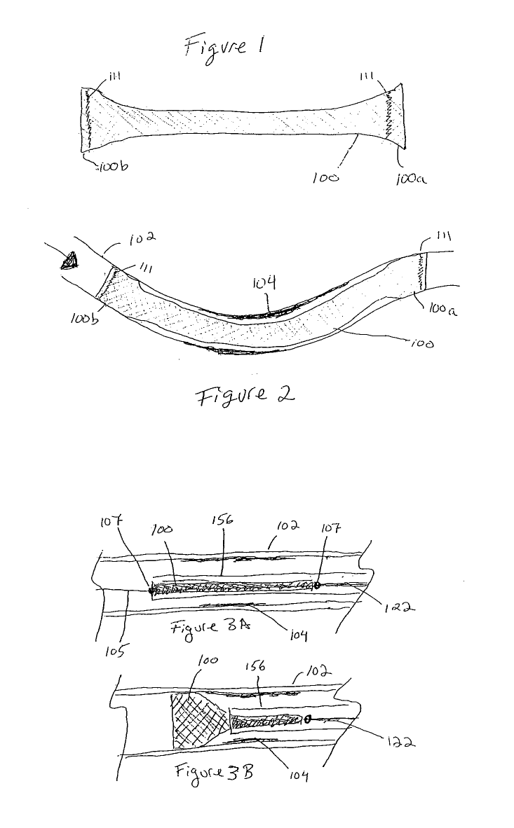

FIG. 1 illustrates one preferred embodiment of a self expanding prosthesis 100 according to the present invention. Unlike prior art prosthesis protectors, the self expanding prosthesis 100 radially expands by its own force, without the need for additional expansion components. This self expanding property allows the self expanding prosthesis 100 to better conform to the inner contours of a vessel 102.

The self expanding force of the self expanding prosthesis 100 is due, in part, to a plurality of filaments coherently engaged together to form a tube shape, for example, by braiding, weaving, or knitting, so as to radially expand in diameter. The filaments may be composed of an elastic metal, polymer, or composite of both, such as nitinol, stainless steel, platinum, or elgiloy and may typically be about 12-25 microns in thickness. In the case of a metal-polymer composite, the polymer may include a pharmacological agent within the polymer structure. Such filaments may also be biostable or biodegradable. Additionally, the biodegradability may be selectively variable to dissolve more rapidly in some areas, such as at branch sites where the filaments may dissolve due to increased blood flow through and around the filaments and thus creating openings for each branch. This concept is illustrated in FIG. 17, which shows a self expanding prosthesis 260 with a dissolved opening 260a created by blood flow into the branch of vessel 102. A scanning electron micrograph of the metal polymer combination can be seen in FIG. 18. In this embodiment, it can be seen that prosthesis 100 has been formed such that it has locations where the filaments are more or less dense than other locations. The less dense locations allow greater blood flow to branch sites that may be located beneath these less dense locations. Over time, these less dense zone of filaments may erode and disappear over time without losing the devices desirable properties in locations outside of the aforesaid side branch.

To achieve the self expanding properties of the self expanding prosthesis, a variety of different combinations of filament diameters, filament components, and engaging styles may be used. Typically, a self expanding prosthesis is annealed on a stainless steel mandrel fixture, which at least partially determines the expanded diameter of the self expanding prosthesis. For example, nitinol may be processed at about 500° C. for about 10-15 minutes with a mandrel of a desired diameter. In another example, stainless steel, Elgiloy, or MP35n materials may be processed at temperatures of about 1000° C. for relatively longer periods such as 2-4 hours. The resulting annealed device will then exhibit a desired expansion force to a desired diameter (again as primarily determined by the mandrel size).

Examples of the structural makeup of a self-expanding prosthesis in accordance with the present invention are listed below. In this regard, these examples reflect primary structural parameters and do not specify a length dimension since these devices can be made to any desired length for the intended purpose.

### EXAMPLE 1

For example, 72 filaments made from 0.0009 inch nitinol wire may be braided with a plain braid setup to create a 90 degree braid angle, ultimately forming a tube with a 4 mm diameter and a pore size of about 250 microns.

### EXAMPLE 2

In another example, 56 filaments made from 0.001 inch stainless steel wire may be braided with a plain braid setup to create a 90 degree braid angle, ultimately forming a tube of 4 mm in diameter with 340 micron pore size and having a higher outward radial force than the previous example.

### EXAMPLE 3

In yet another example, 52 filaments of 0.001 inch stainless steel wire and 4 filaments of 0.0015 inch platinum wire (for radiopacity) may be braided with a plain braid setup to create a 90 degree braid angle, ultimately forming a tube of 4 mm in diameter with about 340 micron pore size and having a radial force higher than the first example.

### EXAMPLE 4

In another example, 0.001 nitinol wire is knit on a 16 needle machine with a 4 mm bore head (defining a 4 mm tube diameter), ultimately creating a tube with 500 micron pore size.

### EXAMPLE 5

In another example, 0.001 stainless steel wire is knit on a 16 needle machine with a 4 mm bore head (defining a 4 mm tube diameter), ultimately creating a tube with 500 micron pore size.

### EXAMPLE 6

In another example, 50 filaments of 0.001 inch nitinol wire may be woven to form a tube of 60 picks per inch and 4 mm in diameter, ultimately creating a tube with 500 micron pore size.

### EXAMPLE 7

In another example, a sputtered nitinol film tube 10-15 microns thick may be used, ultimately creating a tube with 20-40 micron pore size.

### EXAMPLE 8

In yet another example, a sputtered nitinol film tube 10-15 microns thick with micro pleats may be used, ultimately creating a tube with 20-50 micron pore size. These micro pleats 242 (elongated crimps in the prosthesis body) can be seen in FIG. 14 as part of self expanding prosthesis 240, positioned along the axis of the prosthesis 240 for expansion of the diameter of the prosthesis 240. Additionally, the micro pleats 246 may be positioned circumferentially around the prosthesis 244 for expansion in length, as seen in FIG. 15.

### EXAMPLE 9

In another example, a sputtered nitinol film tube 10-15 microns thick with stent laser hole micron pattern system may be used, ultimately creating a tube with 20-50 micron pore size.

### EXAMPLE 10

In another example, a sputtered nitinol film tube 10-15 microns thick with textured mandrel may be used, creating a folding film. Generally with a prosthesis formed from a sputtered film, the sputtered film is sputtered directly onto a mandrel with a textured surface. The textured surface of the mandrel could be, for example, a cross-hatched pattern or a “waffle” type patter. Either way, the patter will create a small “spring zones” in the device that will operate similar to the aforementioned micro pleats and allow the device to flex and expand more readily.

Generally, the number of filaments may vary along the length of the self expanding prosthesis 100 in order to increase or decrease the expansion diameter and expansion force exerted by the self expanding prosthesis 100. Specifically, as the number of filaments increase within a section of the self expanding prosthesis 100, the expansion diameter and radial expansion force both increase. This can be seen in the ends 100a and 100b of self expanding prosthesis 100 which expand outward to a greater diameter than the center section, allowing for a tighter fit at the ends 100a and 100b within a patients vessel 102. Additionally, the radial force of self expanding prosthesis 100 can be increased by including a few larger diameter filaments engaged with relatively smaller sized filaments. In this respect, the overall pore size of the self expanding prosthesis 100 may be kept small, while the outward radial force may be kept relatively high.

The self expanding prosthesis 100 is typically used as a trap to contain plaque 104, particulates, clots, emboli, and other material between the mesh of the self expanding prosthesis 100 and the wall of the vessel 102. FIG. 2 illustrates a typical self expanding prosthesis 100 with flanged ends 100a and 100b within a vessel 102. The self expanding prosthesis 100 is positioned over the plaque 104, creating a pocket that prevents the plaque 104 from being dislodged and traveling through the blood stream.

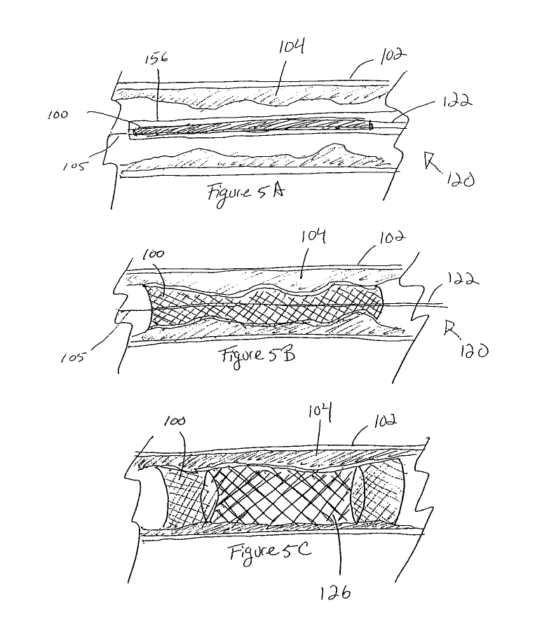

As seen in FIGS. 4A-4C, the self expanding prosthesis 100 may be configured to facilitate growth of tissue 116 (e.g. intima) within and on the surface of the self expanding prosthesis 100. The growth of tissue 116 allows the self expanding prosthesis 100 to permanently trap debris, while creating a new lining to the vessel 102. Further details of the methods used for the growth of such tissue 116 can be found in the co-pending U.S. patent application Ser. No. 09/382,275, entitled Implantable Device For Promoting Repair Of A Body Lumen, filed Aug. 25, 1999, the contents of which are hereby incorporated by reference.

For example, FIG. 4A illustrates a vessel 102 with an ulcerated plaque 112. In FIG. 4B, the self expanding prosthesis 100 is deployed over the ulcerated plaque 112, gently expanding against the walls of vessel 102. As seen in FIG. 4C, over time tissue cells begin to grow into and around the self expanding prosthesis 100, forming a layer of tissue 116 over the self expanding prosthesis 100.

Additionally, the self expanding prosthesis 100 may be used in protecting renal artery dilation (not shown). A proximal end of the self expanding prosthesis 100 is flared to fit the aortic-ostium of the renal artery, while the remainder of the device fits the renal artery. Dilation or stenting is performed in a standard manner, with the self expanding prosthesis 100 in place, allowing for embolic protection, ostial protection, and protection from ostial and renal artery dissections.

If the filaments of the self expanding prosthesis 100 are biostable, the self expanding prosthesis 100 will remain permanently incorporated within the vessel 102. However, if the filaments of self expanding prosthesis 100 are instead composed of biodegradable material, the self expanding prosthesis 100 will gradually break down and disappear, leaving only the new layer of tissue 116. In either respect, the self expanding prosthesis 100 acts to trap dangerous plaque or emboli which may be present, as well as form a new layer of healthy tissue.

Additionally, the filament based material used for the self expanding prosthesis 100 may include a drug coating over a portion or even all of the self expanding prosthesis 100. For example, the self expanding prosthesis 100 may include drugs directed to limit thrombosis, limit neointimal thickening, encourage thin neointima and endothelial coating, limit collagen formation and negative remodeling, limit extracellular matrix formation, and promote collagen growth for containing neointima. The use of the self expanding prosthesis 100 in combination with a drug coating eliminates the need for use of a drug coated stent.

The filament based material may also include anchoring elements (not shown) integrated within the material structure, such as wire hooks, pins, or friction bumps. Once deployed, these elements assist in preventing the self expanding prosthesis 100 from moving from the target location.

The filament based material may also include markers 111, such as radiopaque or platinum filaments woven into the self expanding prosthesis 100. Preferably, the markers 111 are a swaged band positioned at each end of the self expanding prosthesis 100. These markers 111 assist the user in positioning the self expanding deployment device 100 at a desired treatment location.

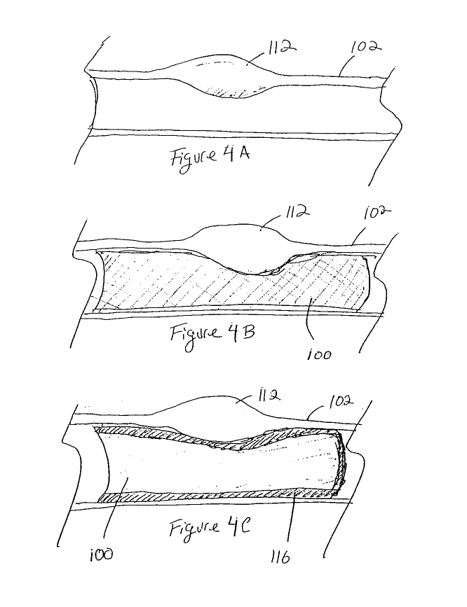

In operation, the self expanding prosthesis 100 is preferably positioned and deployed in a manner similar to a self expanding stent, commonly known in the art. Specifically, as seen in FIGS. 3A and 3B, a guide wire 105 is inserted into the vessel 102 of a patient and advanced to a diseased region of the vessel 102, for example containing plaque 104. Once the guide wire 105 is in a desired target location, a catheter 122 is advanced over the guide wire 105 until the distal end of the catheter 122 is positioned at a desired target location within the vessel 102. The distal end of catheter 122 includes the self expanding prosthesis 100 packed underneath a sheath 156. To assist in positioning the self expanding prosthesis 100 at the diseased location of vessel 102, the catheter 122 includes radiopaque markers 107. When the packed self expanding prosthesis 100 achieves a desired location, the user retracts the sheath 156 in a distal direction (towards the user), exposing the self expanding prosthesis 100. As seen best in FIG. 3B, the self expanding prosthesis 100 is uncovered by the sheath 156, expanding against the vessel 102, trapping plaque 104. Once the self expanding prosthesis 100 has been fully deployed, the user carefully retracts the catheter 122 with the sheath 156, removing them from the patient. In this respect, the self expanding prosthesis 100 acts as a trap for the plaque 104.

Self Expanding Prosthesis With Stent

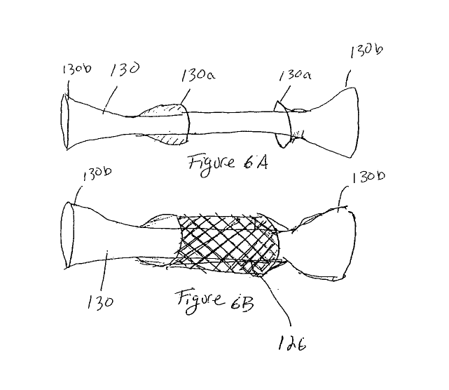

As seen in a preferred embodiment of FIGS. 5A-5C, the self expanding prosthesis 100 may be utilized in conjunction with other cardiovascular treatment devices. For example, a self expanding stent 126 is commonly deployed to increase the diameter of the vessel 102 in a diseased region of the vessel 102 (e.g. plaque 104 buildup causing atherosclerosis). However, deploying a stent 126 to an area of the vessel 102 containing plaque 104 has been shown to create complications resulting from the plaque 102 breaking off and traveling antegrade (downstream) through the blood stream. After breaking off, the plaque 102, also known as emboli, may ultimately block the passage of blood flow to sensitive regions of the body, such as the brain, resulting in stroke or similar organ damage. Therefore, according to the present invention, the self expanding prosthesis 100 may be used to trap the plaque 104, preventing it from breaking off and traveling through the blood stream.

As seen in FIGS. 5A and 5B, the self expanding prosthesis 100 is delivered to a diseased target area of the vessel 102, having a buildup of plaque 104 around the inner surface of the vessel 102. As previously described, a guide wire 105 is positioned at a desired treatment location within the vessel 102. The catheter 122, which contains the self expanding prosthesis 100 packed within the sheath 156, is advanced over the guide wire 105 to the desired treatment region of the vessel 102. The sheath 156 is moved toward the user, in a proximal direction, to expose the self expanding prosthesis 100. The catheter 122 is then removed from the patient and a stent deploying catheter (not shown) is advanced over the guide wire 105 to the same treatment location within the vessel 102. The stent deploying catheter then deploys stent 126 over the self expanding prosthesis 100, expanding the diameter of vessel 102. Since the self expanding prosthesis 100 lies along a longer region of the vessel 102 compared with the stent 126, any plaque 104 that breaks off near the stent 126 is held in position, trapped between the walls of the vessel 102 and the self expanding prosthesis.

Alternately, the present invention may also preferably pack the self expanding prosthesis 100 and the stent 126 onto a single catheter (not shown). For example, this dual deployment may be achieved by compressing the stent 126 over a distal end of the catheter, then compressing the self expanding prosthesis 100 over the stent 126. The distal end of the catheter is finally covered with a sheath (not shown) which prevents both devices from expanding during positioning. Once the catheter is advanced to a desired location, the sheath is drawn back (in a proximal direction), allowing both self expanding prosthesis 100 and stent 126 to expand against a diseased vessel 102.

In another example, a balloon catheter (not shown) may be used to deploy the stent 126 and self expanding prosthesis 100. The stent 126 is compressed over the catheter balloon (not shown), followed by compression of the self expanding prosthesis 100 on top of the stent 126. To maintain the compressed state of both devices, a plurality of wires, fibers, or other string-like filaments encircle the distal end of the catheter, over the self expanding prosthesis 100. Thus, once the distal end of the catheter is transported to a desired treatment area within the vessel 102, the catheter balloon is inflated, causing the filaments encircling both devices to break. With no restraints holding them in a compressed state, the self expanding prosthesis 100 and subsequently the stent 126 radially expand against the inner walls of the vessel 102. In addition to the benefit of deploying both devices at once, the user may optionally utilize the catheter balloon for additional treatment purposes.

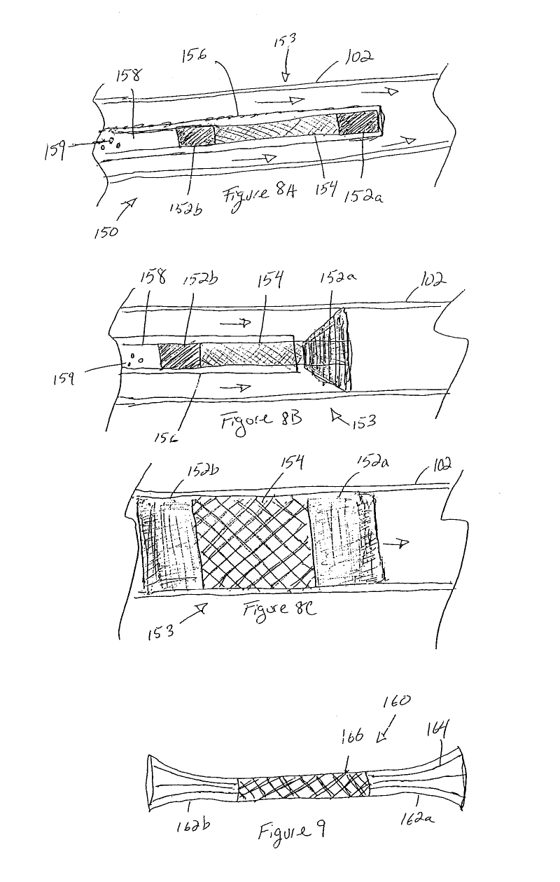

Referring to FIGS. 7A-7C, another preferred embodiment is illustrated in accordance with the present invention. Specifically, a self expanding prosthesis 142 and spiral stent 146 are shown which allow both of the ends 142b of the self expanding prosthesis 142 to expand prior to expansion of the stent 146. This differing expansion, best seen in FIG. 7B, may be accomplished by using two distinct methods to control expansion of the self expanding prosthesis 142 and the stent 146.

For example, the self expanding prosthesis 142 is compressed on a catheter 144. The stent 146 is further positioned and compressed on top of the self expanding prosthesis 142, centered to allow an equal amount of the self expanding prosthesis device 142 (e.g. ends 142a) to extend past the stent 146 on each end. The stent 146 is held in place by a trigger wire (not shown) which wraps around the stent 146 and further passes down a lumen in the catheter 144, allowing a user pull the trigger wire to release the stent 146 to its expanded shape. The ends 142a, however, are maintained in a compressed position by a sheath (not shown).

In operation, the user positions the guide wire 105 at a desired target location within a vessel 102. The catheter 144 is advanced over the guide wire 105 to the target location. Next, the user draws back the sheath in a proximal direction (toward the user), exposing both the self expanding prosthesis 142 and stent 146. Since the stent 146 is still constricted by the trip wire, only the ends 142a of self expanding prosthesis 142 expand radially outward, as seen in FIG. 7B. Finally, the user pulls the trip wire, releasing the stent 146 to expand against the vessel 102. In this respect, the ends 142a function as a initial barriers, trapping any plaque 102 or other debris that may dislodge during the procedure.

Self Expanding Prosthesis With Stent Pockets

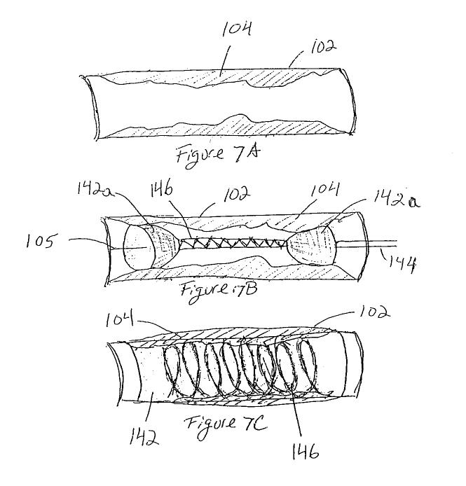

Referring now to FIGS. 6A and 6B, another embodiment of the present invention is illustrated. The self expanding prosthesis 130 is similar to the previously described embodiments of this application, yet further includes stent pockets 130a for capturing and maintaining a stent 126. The stent pockets 130a are composed of the same filament material as the body of self expanding prosthesis 130, allowing the pockets 130a to stretch longitudinally to accommodate the stent 126.

It is preferred that the ends 130b of the self expanding prosthesis 130 flare radially outward, as previously described elsewhere in this application, such as in reference to FIGS. 1 and 2. Since the stent pockets 130a maintain the stent 126 around the outer diameter of self expanding prosthesis 100, the flared ends 130b ensure that dislodged plaque (not shown) or other emboli do not escape from underneath the self expanding prosthesis 130. In this respect, a pocket is formed between the self expanding prosthesis device 130 and the vessel walls (not shown in FIGS. 6A and 6B), enclosing both the stent 126 and any plaque (not shown in FIGS. 6A and 6B) also present.

The self expanding prosthesis 130 and the stent 126 may be delivered to a target location as a single device (i.e. with the stent engaged with the stent pockets 130a). The delivery could be performed by a variety of techniques, such as the previously described method utilizing a sheath to maintain the self expanding prosthesis 130 and stent 126 in a compressed state.

In another preferred embodiment (not shown), the self expanding prosthesis may include a single elongated stent pocket. A single stent pocket may provide less material than two stent pockets, allowing the self expanding prosthesis to more closely expand against a vessel wall.

Stent With Self Expanding End Filters

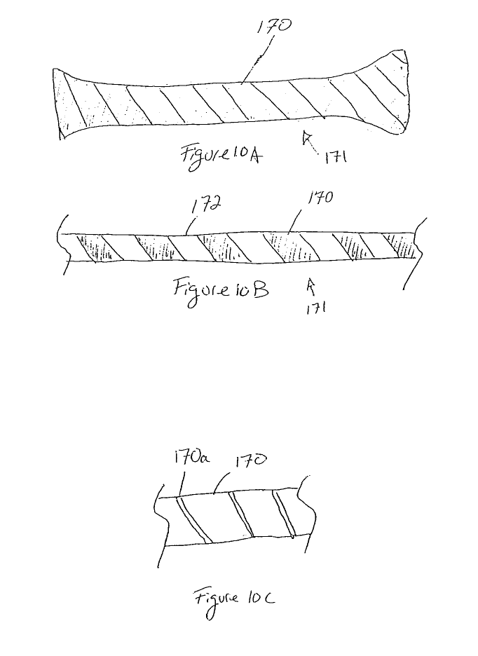

FIGS. 8A-8C illustrate yet another preferred embodiment of the present invention. A filtering stent 153 includes a center stent portion 154 having two self expanding end sections 152a and 152b coupled to the center stent portion 154. The self expanding end sections 152a and 152b may be composed of the same material described elsewhere in this application for the varying embodiments of the self expanding prostheses (e.g. the prostheses 100 as seen in FIGS. 1 and 2).

The stent portion 154 is similar to a self expanding stent composed of braided nitinol fibers, however any number of stent-like designs similar to those known in the art may be used. The self expanding end sections 152a and 152b may be coupled to the stent portion 154 by welding, interweaving, interbraiding, or integral forming. Preferably, the self expanding end sections 152a and 152b are at least about the length of the internal diameter of the end sections 152a and 152b when expanded, however lengths may also be longer. In a preferred embodiment, when expanded, the end sections will generally resemble a square or horizontal rectangle shape.

As seen in FIG. 8A, the filtering stent 153 is preferably inserted into a vessel 102 upstream of a desired treatment site, as seen by the arrows representing blood flow. The filtering stent 153 is compressed around a distal end of a delivery catheter 158 and maintained in said compressed state by the sheath 156. When the filtering stent 153 has achieved a desired target position within vessel 102, the sheath 156 is retracted proximally towards the user, as seen in FIG. 8B. As the sheath 156 retracts, it first exposes self expanding end section 152a which expands radially in diameter against the walls of vessel 102. The sheath 156 is drawn back further from the distal end of the catheter 158, fully exposing filtering stent 153 and allowing the filtering stent 153, including stent portion 154 and self expanding end section 152b, to expand in diameter against the walls of vessel 102.

The self expanding end section 152a functions as an integrated filter downstream of the stent portion 154. Thus, as the stent portion 154 expands and dislodges debris within the vessel 102, self expanding end section 152a catches this debris, ultimately holding it against the walls of vessel 102. In this respect, the debris is prevented from passing downstream, causing additional and possibly serious complications. The self expanding end section 152b deploys last and may, for example, prevent plaque to move in a retrograde direction due to currents created by the deploying filtering stent 153.

In another preferred embodiment, the self expanding end section 152b is not present on the filtering stent 153, since it is deployed last, retrograde to the stent portion 154 and therefore does not filter antegrade to the stent portion 154.

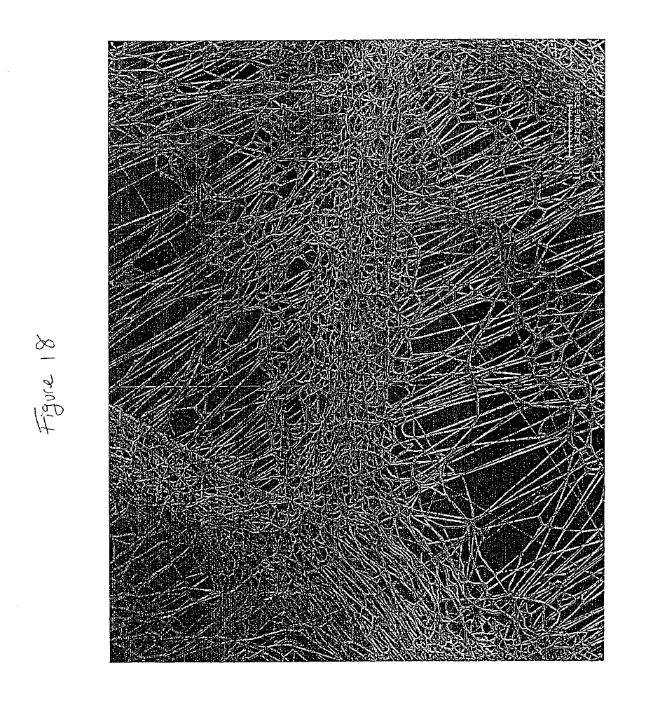

In yet another preferred embodiment seen in FIG. 16, a tapered self expanding end section 152c is included at the distal end of the stent portion 154. The tapered self expanding section 152c is similar to self expanding end section 152a of FIGS. 8a-8c, however, end section 152c is compressed to a tapered shape to facilitate position within a vessel 102. Typically, the stent portion 154 compresses to a diameter of about 3 French, while the self expanding end sections 152a, 152b, 152c (as well as other self expanding embodiments described in this application) may compress to a diameter of about 2 French or smaller. Thus, a tapered shape of end section 152c may be achieved by, for example, utilizing a trip wire (not shown) to pack the end section 152.

As seen in FIGS. 8A and 8B, the filter stent 153 includes contrast ports 159, located on the body of catheter 158, proximal to the filtering stent 153. The contrast ports 159 are in fluid communication with a lumen within the catheter 158, which may be connected to a supply of contrast media. Once self expanding end section 152a and/or 152b is deployed to form an angled funnel shape, the contrast media may be introduced into the body lumen through the catheter ports 159 and thereafter travel through the small porosity of either of the end sections 152a, 152b, thereby improving the ability to visualize the location of the filter stent 153. Note, the contrast ports 159 of this preferred embodiment may also be used with the other preferred embodiments of this application.

FIG. 9 illustrates a preferred embodiment according to the present invention of a filtering stent 160 having struts 164 longitudinally positioned around the diameter of the filter stent 160. The filtering stent 160 is generally similar to the previously described filtering stent 153, having a center stent portion 166 coupled to two self expanding ends 162a and 162b. However, the filtering stent 160 also includes the struts 164 which assist in the expansion and overall conformation of the filtering stent 160. For example, the struts 164 may be radially angled outward from the filtering stent 160, creating a flare in the self expanding end sections 162a and 162b. Preferably, the struts are composed from an elastic metal or flexible polymer with a preconfigured shape, allowing the struts to flatten out and compress with the filtering stent 160 when packed within a deployment catheter.

Self Expanding Ribbon Prosthesis

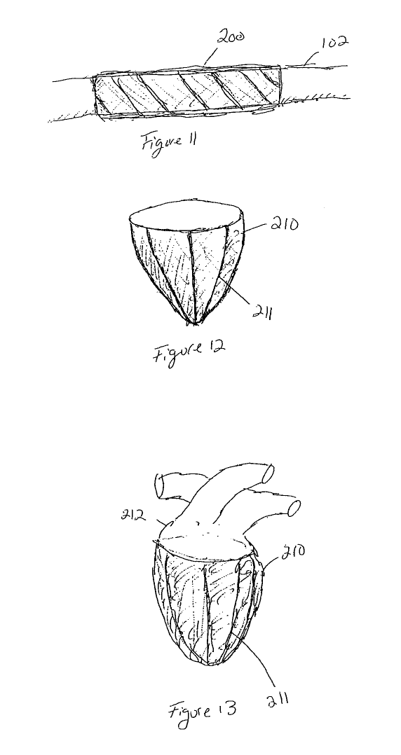

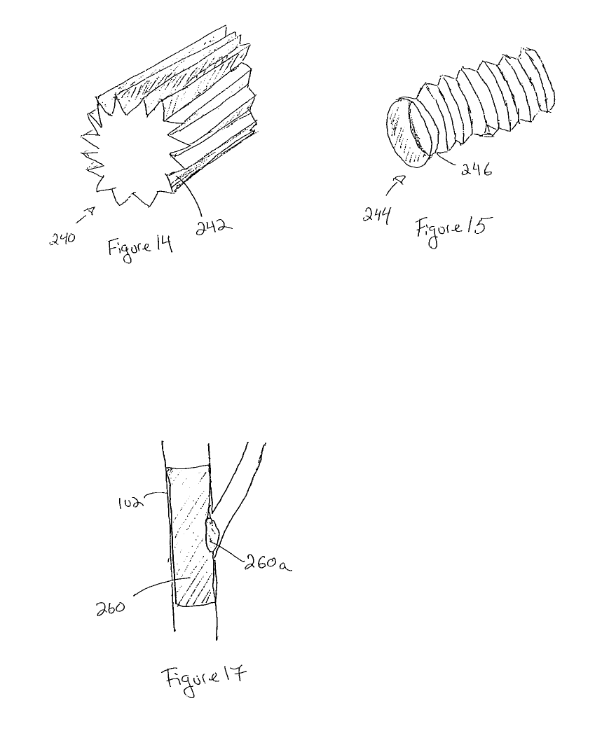

FIGS. 10A-10C illustrate a self expanding ribbon prosthesis 171 according to the present invention. The self expanding ribbon prosthesis 171 is similar in overall expanded shape and material to the self expanding prosthesis embodiments described elsewhere in this application (e.g. self expanding prosthesis 100 of FIGS. 1 and 2), however, the self expanding ribbon prosthesis 171 is formed from a length of ribbon 170 which is preconfigured to curve around to form a tube, as seen in FIG. 10A. The self expanding ribbon prosthesis 171 is preferably made from Nitinol woven, braided, or knitted fabric, similar to the previous embodiments described in this application. For example, 0.0005-0.0009 inch diameter Nitinol wire may be used (Elgiloy, MP35n or other similar wire may also be used), creating an overall tube shape when expanded with a width of about 3-6 mm.

The self expanding ribbon prosthesis 171 maintains a cohesive tube form when in an expanded position by forming overlapping circular loops of ribbon 170, best seen in FIG. 10C. Thus, when the self expanding ribbon prosthesis 171 expands, no gaps remain between the curls of ribbon 170, allowing the self expanding ribbon prosthesis 171 to hold plaque and other debris against a vessel wall (not shown in FIGS. 10A-10C).

In operation, the self expanding ribbon prosthesis 171 is compressed and wound around a delivery catheter 172, as seen in FIG. 10B. Since the ribbon 170 is configured to expand to a larger diameter than the delivery catheter 172, the ribbon 170 will spread out along the catheter 172 in a non-overlapping layout. The ribbon 170 is maintained in a compressed state on the catheter 172 by a sheath (not shown) positioned over the ribbon, 170, however, alternative compression techniques may be used also, such as a trigger wire (not shown) wrapped around the ribbon 170 and releasable by the user.

As with previous embodiments described in this application, a distal end of the delivery catheter is positioned within a patient at a desired treatment location (e.g. within a vessel). Once in place, ribbon 170 is released from the catheter 172, expanding in height, while compressing in length until the curls of ribbon 170 overlap each other and press against the wall of the vessel. Thus, the self expanding ribbon prosthesis 171 functions similarly to the prosthesis of FIGS. 1 and 2 to prevent plaque, debris, emboli, clots, and other material from dislodging and causing complications downstream. As with previously described embodiments in this application, the self expanding ribbon prosthesis 171 may be used with other treatments, such as a stent or catheter balloon.

External Self Compressing Prosthesis

FIG. 11 illustrates yet another preferred embodiment according to the present invention. An external self compressing prosthesis 200 has a generally ribbon-like structure, similar in overall structure and material to the self expanding ribbon prosthesis 171 shown in FIGS. 10A-10C. However, the external self compressing prosthesis 200 is structured to contract instead of expand, allowing the external self compressing prosthesis 200 to conform to an external organ for treatment purposes, such as the vessel 102 seen in FIG. 11.

For example, the external self contracting prosthesis 200 may be positioned around a vessel 102 after a vascular incision has been made. The material of external self contracting prosthesis 200 may be structured to facilitate cellular ingrowth, as previously described in this application. Thus, with a compatible porosity, the external self contracting prosthesis 200 develops a neo-adventitia. Additionally, drugs may be included to elute from the external self contracting prosthesis 200 for a variety of different treatment purposes, for example to limit hyperplasia, provide anti-thrombotic effects, promote adventitial organized and beneficial cellular ingrowth, promote adventitial neovascularization, promote a neoadventitia, limit adventitial scarring, or inhibit adventitial neovascularization.

The material of external self contracting prosthesis 200 may be bioabsorbable with a programmable dissolution rate, preferably programmed to dissolve after cellular growth has sufficiently infiltrated the prosthesis 200 to remain intact of its own accord. Additionally, the prosthesis 200 may be anchored to the organ by way of needles, hooks, brief electrical energy burst coagulating proteins or other biological molecules to the surface of the prosthesis 200, adhesive substances, or other anchoring methods.

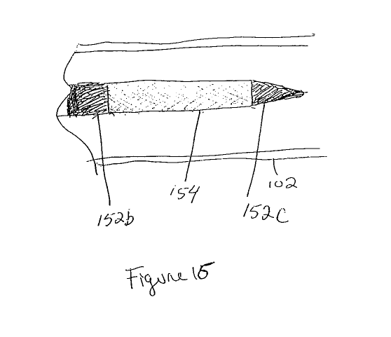

In addition to tube shapes, the self contracting prosthesis may be formed to a number of shapes, such as the heart prosthesis 210 seen in FIGS. 12 and 13. The heart prosthesis 210 may be used for many potential heart 212 treatments, such as constraining the size of a heart 212 to prevent a specific growth size or drug delivery. For example, potential drugs may include statins, anti-inflammatory agents, anti-platetet (including antibodies such as Gp IIb/IIIa antibody), substances to dissolve calcium or lipids, or matrix metalloprotease. As with previously described embodiments of this application, struts 211 may be included for structural and contracting support.

The heart prosthesis 210 is preferably delivered percutaneously, preloaded in an inverted position within a delivery catheter (not shown). The a distal end of the delivery catheter is placed near the apex of the heart 212 within the pericardial space while the user deploys the heart prosthesis 210, unrolling the heart prosthesis 210 over the heart 212.

The heart prosthesis 210 may include additional functionality such as one or more electrical conductive regions that are connectable to pacing leads, creating an epicardial system. Multiple pacing lead targets may be present but not used, providing a left or right ventricular electrode set, selectable for the best leads. The heart prosthesis 210 may also include multiple epicardial pacing sites which can be synchronized together to minimize the effective QRS complex width.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A prosthesis for trapping undesired particles in a body lumen comprising:
a generally tubular body having a contracted state and an enlarged state;
said generally tubular body being comprised of a plurality of microfilaments that interconnect to create a pore size no greater than about 500 microns substantially along the length of said generally tubular body;
said generally tubular body being self expandable from said contracted state to said enlarged state; and,
said generally tubular body being sufficiently flexible such that said tubular body conforms to a contour of an inner surface of said body lumen.

2. A prosthesis according to claim 1, wherein said plurality of microfilaments comprises a plurality of woven microfilaments.

3. A prosthesis according to claim 1, wherein said plurality of microfilaments comprises a plurality of braided microfilaments.

4. A prosthesis according to claim 1, wherein said plurality of microfilaments comprises a plurality of knitted microfilaments.

5. A prosthesis according to claim 1, wherein said plurality of microfilaments comprises a plurality of sputtered microfilaments

6. A prosthesis according to claim 1, wherein said generally tubular body has two ends, at least one of which being expandable to a greater diameter than a central region of said generally tubular body.

7. A prosthesis according to claim 6, wherein said at least one end has a flared shape in said enlarged state of said tubular body.

8. A prosthesis according to claim 1, wherein said microfilaments are bioresorbable.

9. A prosthesis according to claim 8, wherein said microfilaments are bioresorbable such that increased blood flow through said microfilaments at a location of a lumen side branch accelerates the rate of bioresorbtion of said micrcofilaments at said location.

10. A prosthesis according to claim 1, wherein generally tubular body is at least partially loaded with a drug.

11. A prosthesis according to claim 1, wherein a distal end of said generally tubular body has a cone shape when said generally tubular body is in said contracted state.

12. A prosthesis according to claim 1, wherein said generally tubular body includes a plurality of micropleats.

13. A prosthesis according to claim 12, wherein said micropleats extend longitudinally along an axis of said generally tubular body.

14. A prosthesis according to claim 12, wherein said micropleats extend circumferentially along an axis of said generally tubular body.

15. A prosthesis according to claim 1, wherein said generally tubular body in said contracted state has a ribbon configuration wherein gaps exist between curls of said ribbon and wherein said generally tubular body in said expanded state has a ribbon configuration wherein no gaps exist between said curls of said ribbon.

16. A prosthesis according to claim 1, further comprising a stent disposed internally to said generally tubular body.

17. A prosthesis according to claim 16, wherein said stent is integral with said generally tubular body.

18. A prosthesis according to claim 16, wherein said a length of said generally tubular body is longer than said stent.

19. A prosthesis according to claim 16, wherein said generally tubular body and said stent are constrained in said contracted state with breakable filaments.

20. A prosthesis according to claim 1, further comprising at least one pocket disposed circumferentially on said generally tubular body, said pocket sized to receive a stent.
